(19) United States
(12) Patent Application Publication
(10) Pub. No.: US 2024/0226876 A1
(43) Pub. Date: Jul. 11, 2024

(54) TESTING DEVICES

(71) Applicants: THE REGENTS OF THE UNIVERSITY OF CALIFORNIA, Oakland, CA (US); CHAN ZUCKERBERG BIOHUB, INC., San Francisco, CA (US)

(72) Inventors: Adam Abate, Oakland, CA (US); Charles Chiu, Oakland, CA (US); Krzysztof Langer, Oakland, CA (US); Daniel Weisgerber, Oakland, CA (US)

(21) Appl. No.: 18/288,356

(22) PCT Filed: Apr. 25, 2022
(86) PCT No.: PCT/US22/26191

§ 371 Date: Oct. 25, 2023

Related U.S. Application Data
(60) Provisional application No. 63/180,051, filed on Apr. 26, 2021.

(51) Int. Cl. B01L 3/00; B01L 7/00

(52) U.S. Cl. CPC B01L 3/5027; B01L 7/52; B01L 2300/0883; B01L 2300/1827

(57) ABSTRACT

Testing devices are disclosed. In accordance with a first implementation, a testing device includes a sample chamber is to receive a sample and a reagent reservoir that contains a reagent used to determine a presence of a target molecule in the sample. A sample fluidic line is fluidically coupled to the sample chamber and a common fluidic line and a reagent fluidic line is fluidically coupled to the reagent reservoir and to the common fluidic line. A diagnostic indicator is coupled to the common fluidic line. The sample and the reagent flow through the respective sample fluidic line and the reagent fluidic line toward the common fluidic line and form a mixture within the common fluidic line and the common fluidic line enables a threshold incubation period of the mixture prior to the mixture flowing to the diagnostic indicator.

### RELATED APPLICATION

This application claims the benefit of and priority to U.S. Provisional Patent Application No. 63/180,051, filed Apr. 26, 2021, the content of which is incorporated by reference herein in its entireties and for all purposes.

### FIELD OF THE DISCLOSURE

The present patent relates generally to testing devices and, in particular, to testing devices for at-home and/or point-of-care diagnostics.

### BACKGROUND

At-home testing devices may be used to test for different target molecules.

### SUMMARY

At least one aspect of this disclosure is directed toward testing devices and related systems for at-home and/or point-of-care diagnostics that can perform molecular tests to identify one or more target molecules of interest. The disclosed testing devices may be modular and produced/sold at a relatively low cost. The disclosed testing devices can also be used in clinical-settings and/or in field settings, such as air ports and/or community centers, to process dozens to hundreds of samples in a timely fashion.

A wide spectrum of molecular tests may be performed using the disclosed examples including tests for severe acute respiratory syndrome coronavirus 2 [COVID-19], Human severe acute respiratory syndrome SARS coronavirus, respiratory syncytial virus, adeno-associated virus, zika virus, an influenza A virus, an influenza B virus, an influenza C virus, a human immunodeficiency virus, Hepatitis B, Hepatitis C, or cancer. In implementations in which tests performed are for Covid-19, these tests may be routinely performed (e.g., daily) to determine the presence/absence of the virus.

In some implementations, a testing kit may be provided that includes the testing device and a swab used to perform a sampling procedure. The sampling procedure may include a nasal swab, a nasopharyngeal swab, a throat swab, or a saliva sample.

The testing device includes a sample chamber to receive the sample, a reagent reservoir containing reagent to determine a presence of a target molecule in the sample, a diagnostic indicator, and fluidic lines that fluidically couple the sample chamber, the reagent reservoir, and the diagnostic indicator. In some implementations, the fluidic lines include a sample fluidic line, a reagent fluidic line, and a common fluidic line, where the sample fluidic line fluidically couples the sample chamber and the common fluidic line and the reagent fluidic line fluidically couples the reagent reservoir and the common fluidic line. The testing device also includes a pump that is used to urge the sample through the sample fluidic line and toward the common fluidic line and urge the reagent(s) through the reagent fluidic line and toward the common fluidic line. In other implementations, the testing device includes a consumable that is used with a system that includes the pump. In such implementations, the system may include a receptacle to receive the consumable, the pump is used to urge the sample and/or reagent through the corresponding fluidic lines, and a heater is used to heat one or more of the sample fluidic line, the reagent fluidic line, or the common fluidic line.

To obtain a sample for testing using the testing kit assembly, an individual can perform a nasal swab, a nasopharyngeal swab, or a throat swab to obtain a sample, which is placed into the sample chamber. Alternatively, a saliva sample may be obtained. A viral transport media (VTM) and/or a buffer may be added to or be present in the sample chamber. The pump then urges the sample/buffer mixture through the sample fluidic line toward the common fluidic line and urges the reagent(s) through the reagent fluidic line toward the common fluidic line to allow the sample/buffer mixture and the reagent(s) to mix. The common fluidic line may include a passive mixer such as a herringbone mixer that is used to mix the sample, buffer, and reagent together. The common fluidic line enables the mixture to incubate for a threshold period of time prior to the mixture flowing to the diagnostic indicator, where the presence of the target molecule can be detected using, for example, a litmus-style display and/or imaging system.

While the above example mentions a single target being detected, the testing devices may allow for microfluidic parallelization and/or the presence of multiple targets to be detected. To do so, additional reagent reservoirs containing additional reagents, diagnostics indicators, and/or fluidic lines may be provided. The disclosed implementations can be used in conjunction with other known detection methods, including, for example, the method described in the following article: https://www.medrxiv.org/content/10.1101/2021.11.29.21267041v1.

In accordance with a first implementation, a testing device includes a sample chamber, a reagent reservoir, a sample fluidic line, a reagent fluidic line, a common fluidic line, and a diagnostic indicator. The sample chamber is to receive a sample and the reagent reservoir contains a reagent used to determine a presence of a target molecule in the sample. The sample fluidic line is fluidically coupled to the sample chamber and the common fluidic line and the reagent fluidic line is fluidically coupled to the reagent reservoir and to the common fluidic line. The diagnostic indicator is coupled to the common fluidic line. The sample and the reagent flow through the respective sample fluidic line and the reagent fluidic line toward the common fluidic line and form a mixture within the common fluidic line. The common fluidic line enables a threshold incubation period of the mixture prior to the mixture flowing to the diagnostic indicator.

In further accordance with the foregoing example, an apparatus and/or method may further include any one or more of the following:

In accordance with one example, the reagent includes a Loop-mediated Isothermal Amplification (LAMP) reagent.

In accordance with another example, the reagent includes a polymerase chain reaction (PCR) reagent.

In accordance with another example, the reagent includes one or more of a lysis reagent, a primer reagent, a polymerase reagent, a deoxynucleotide triphosphate (dNTPs) reagent, and a buffer reagent.

In accordance with another example, the sample is associated with a saliva sample.

In accordance with another example, the sample is associated with a nasopharyngeal swab sample.

In accordance with another example, the target molecule is a nucleic acid.

In accordance with another example, the nucleic acid is DNA or RNA.

In accordance with another example, the DNA or RNA is from a pathogen selected from the group consisting of a virus, a bacteria, a fungi, a protist, and a parasite.

In accordance with another example, the pathogen is a coronavirus.

In accordance with another example, the pathogen is associated with one or more of severe acute respiratory syndrome coronavirus 2, Human severe acute respiratory syndrome SARS coronavirus, respiratory syncytial virus, adeno-associated virus, zika virus, an influenza A virus, an influenza B virus, an influenza C virus, a human immunodeficiency virus, Hepatitis B, Hepatitis C, or cancer.

In accordance with another example, the common fluidic line is at least one of sized or shaped to enable the threshold incubation period.

In accordance with another example, the common fluidic line includes a serpentine channel that enables the threshold incubation period.

In accordance with another example, the incubation period is approximately five minutes.

In accordance with another example, the common fluidic line includes a passive mixer.

In accordance with another example, the passive mixer includes a herringbone mixer.

In accordance with another example, the testing device includes a heater positioned to heat one or more of the sample fluidic line, the reagent fluidic line, or the common fluidic line.

In accordance with another example, the heater includes thermoelectric tape.

In accordance with another example, the heater includes a flexible heater.

In accordance with another example, the heater is positioned to heat at least a portion of the sample fluidic line, a portion of the reagent fluidic line, and a portion of the common fluidic line.

In accordance with another example, the testing device includes a power source operatively coupled to the heater.

In accordance with another example, the testing device includes a first heater positioned to heat at least a portion of the sample fluidic line and a second heater positioned to heat a portion of the common fluidic line.

In accordance with another example, the testing device includes a pump to urge the sample from the sample chamber toward the common fluidic line and to urge the reagent from the reagent reservoir toward the common fluidic line.

In accordance with another example, the pump includes a piston pump.

In accordance with another example, the testing device includes a body defining a bore, the piston pump includes a piston and the bore, and the piston is slidably disposed within the bore.

In accordance with another example, the testing device includes a lid hingably coupled to the body and movable between an open position and a closed position. The lid is to move the piston within the bore when the lid moves toward the closed position.

In accordance with another example, the sample chamber and the reagent reservoir include corresponding openings and the lid seals against the openings when the lid is in the closed position to allow the sample chamber and the reagent reservoir to be pressurized by the piston pump.

In accordance with another example, the sample chamber comprises an opening and the lid seals against the opening when the lid is in the closed position to allow the sample chamber to be pressurized by the piston pump.

In accordance with another example, the testing device includes a hydrophobic venting membrane covering an opening of the reagent reservoir.

In accordance with another example, the reagent includes a dry reagent.

In accordance with another example, the reagent includes a liquid reagent.

In accordance with another example, the testing device includes a buffer within the sample chamber.

In accordance with another example, the testing device includes a second reagent reservoir, a second reagent fluidic line, a second common fluidic line, and a second diagnostic indicator. The second reagent reservoir contains a second reagent used to determine a presence of a second target molecule in the sample. The sample fluidic line is fluidically coupled to the sample chamber and the second common fluidic line and the second reagent fluidic line is fluidically coupled to the second reagent reservoir and to the second common fluidic line. The second diagnostic indicator is coupled to the second common fluidic line.

In accordance with another example, the testing device includes a consumable including the sample chamber, the reagent reservoir, the sample fluidic line, the reagent fluidic line, and the common fluidic line. The testing device also includes a system including a receptacle, a pump, and a heater. The receptacle is to receive the consumable and the pump is fluidically couplable to one or more of the sample chamber, the reagent reservoir, the sample fluidic line, or the reagent fluidic line. The heater is positioned to heat one or more of the sample fluidic line, the reagent fluidic line, or the common fluidic line. The pump urges the sample from the sample chamber toward the common fluidic line and urges the reagent from the reagent reservoir toward the common fluidic line.

In accordance with another example, the testing device includes a user interface to display results associated with the presence of the target molecule within the sample or the target molecule not being present within the sample.

In accordance with another example, the system includes a communication interface to enable communication between the system and a remote system.

In accordance with another example, the communication is associated with the presence of the target molecule in the sample.

In accordance with another example, the communication interface is a wireless interface.

In accordance with another example, the communication interface is to communicate using a short-range wireless communication.

In accordance with another example, the diagnostic indicator includes a visual indicator.

In accordance with another example, the testing device comprises an at-home testing device.

In accordance with another example, each of the sample fluidic line, the reagent fluidic, and the common fluidic line comprise microfluidic lines.

In accordance with another example, the testing device comprises a microfluidic device.

In accordance with another example, the testing device includes a droplet generator.

In accordance with another example, the droplet generator includes a fluidic line coupled to one or more of the sample fluidic line, the reagent fluidic line, and the common fluidic line.

In accordance with another example, the fluidic line contains a continuous non-aqueous phase liquid.

In accordance with another example, the continuous non-aqueous phase liquid includes a continuous non-aqueous phase oil with surfactant.

In accordance with another example, the reagent and the continuous non-aqueous phase oil with surfactant form a fluorescent droplet emulsion.

### BRIEF DESCRIPTION OF THE DRAWINGS

FIG. 1 illustrates a schematic diagram of an implementation of a testing device in accordance with the teachings of this disclosure.

FIG. 2 is a top plan view of an example implementation of a portion of the testing device of FIG. 1.

FIG. 3 is a detailed view of the passive mixer of the sample fluidic line of the testing device FIG. 2.

FIG. 4 is a detailed view of the intersection between the sample fluidic line and the reagent fluidic line of the testing device of FIG. 2.

FIG. 5 is a detailed view of the passive mixer of the common fluidic line of the testing device of FIG. 2.

FIG. 6 is an isometric view of another implementation of the testing device of FIG. 1 and a swab that is used to obtain a sample from an individual.

FIG. 7 is a process diagram including processes used to perform a testing operation using the testing device in accordance with the teachings of this disclosure.

FIG. 8 is a schematic diagram of an implementation of another testing device in accordance with the teachings of this disclosure.

FIG. 9 is a schematic diagram of an implementation of another testing device in accordance with the teachings of this disclosure.

FIG. 10 is a schematic diagram of an implementation of another testing device in accordance with the teachings of this disclosure.

### DETAILED DESCRIPTION

Although the following text discloses a detailed description of example methods, apparatus and/or articles of manufacture, it should be understood that the legal scope of the property right is defined by the words of the claims set forth at the end of this patent. Accordingly, the following detailed description is to be construed as examples only and does not describe every possible example, as describing every possible example would be impractical, if not impossible. Numerous alternative examples could be implemented, using either current technology or technology developed after the filing date of this patent. It is envisioned that such alternative examples would still fall within the scope of the claims.

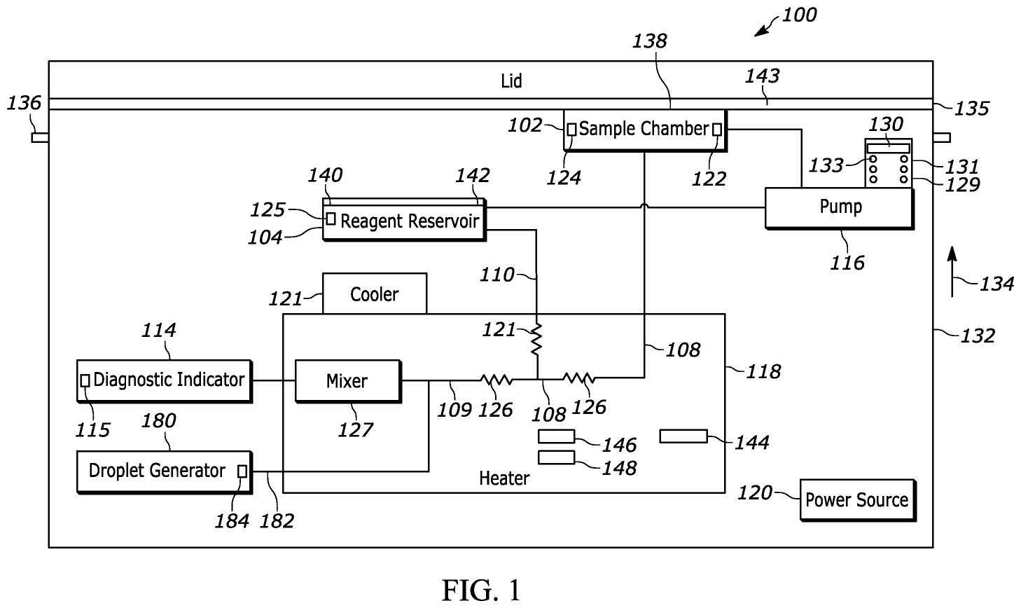

FIG. 1 illustrates a schematic diagram of an implementation of a testing device 100 in accordance with the teachings of this disclosure. The testing device 100 can be used to perform an analysis on one or more samples of interest. In the implementation shown, the testing device 100 is a hand-held device that can be used for at-home diagnostics and/or point-of-care diagnostics and includes a sample chamber 102 and a reagent reservoir 104. The testing device 100 also includes a sample fluidic line 108 that is fluidically coupled to the sample chamber 102 and a common fluidic line 109, and a reagent fluidic line 110 that is fluidically coupled to the reagent reservoir 104 and the common fluidic line 109.

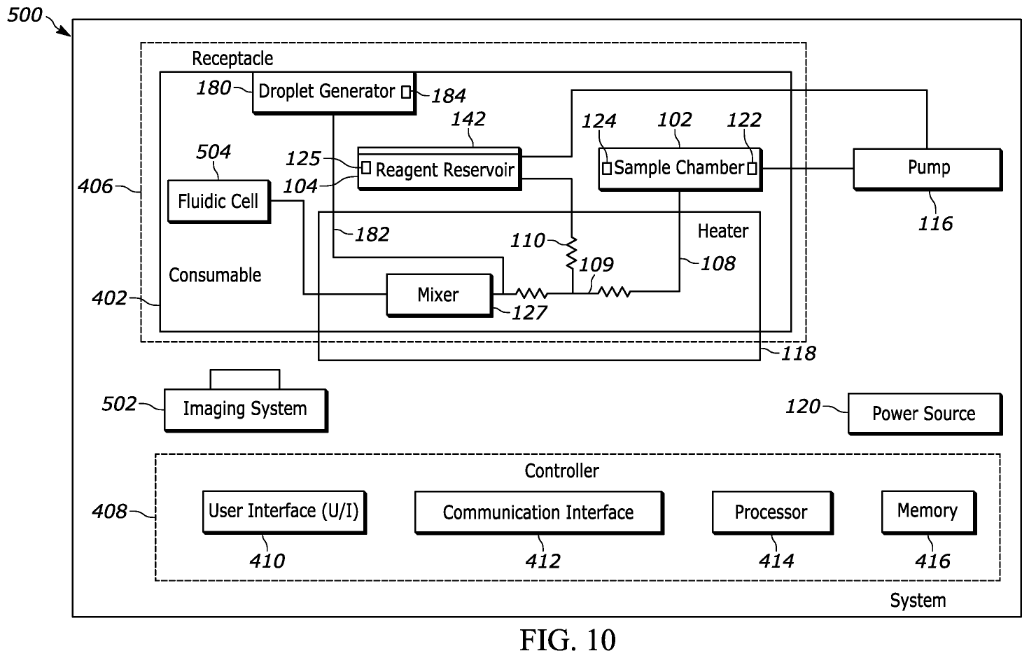

The testing device 100 also includes a diagnostic indicator 114 that may be used to indicate the presence of a target molecule which may be a nucleic acid such as DNA or RNA. The DNA or RNA can be from a pathogen selected from the group consisting of a virus, a bacteria, a fungi, a protist, and a parasite. In some implementations, the pathogen is a coronavirus and/or associated with one or more of severe acute respiratory syndrome coronavirus 2, Human severe acute respiratory syndrome SARS coronavirus, respiratory syncytial virus, adeno-associated virus, zika virus, an influenza A virus, an influenza B virus, an influenza C virus, a human immunodeficiency virus, Hepatitis B, Hepatitis C, or cancer. The diagnostic indicator 114 may include a litmus-style display 115 and/or a visual indictor. However, in implementations in which an imaging system is used to determine the presence of a target molecule of interest, the diagnostic indicator 114 may be omitted and replaced with, for example, a fluidic channel and/or a flow cell (see, for example, FIG. 10).

Referring still to the testing device 100, in the implementation shown, the testing device 100 includes a pump 116, a heater 118, and a power source 120 operatively coupled to the heater 118. The pump 116 is fluidically coupled to the sample chamber 102 and the reagent reservoir 104 and the heater 118 is positioned to heat one or more of the sample fluidic line 108, the reagent fluidic line 110, or the common fluidic line 109. In some implementations, the testing device 100 includes a cooler 121 positioned to cool one or more of the sample fluidic line 108, the reagent fluidic line 110, or the common fluidic line 109. The cooler 121 may be a peltier plate and/or a peltier cooler and the one or more of the sample fluidic line 108, the reagent fluidic line 110, or the common fluidic line 109 may be positioned between the heater 118 and the cooler 121. Alternatively, the cooler 121 may be omitted. While not shown, the testing device 100 may also include one or more valves, such as a rotary valve, a pinch valve, a flat valve, a solenoid valve, a check valve, a piezo valve, and/or a three-way valve. However, different types of fluid control devices may be used. The valves may be actuated using components of the testing device 100 and/or in response to a lid of the testing device 100 being closed.

To perform a testing operation, a sample 122 can be added to the sample chamber 102 that may include a buffer 124 such as a lysis buffer. The reagent reservoir 104 may include a reagent 125 such as a Loop-mediated Isothermal Amplification (LAMP) reagent, a polymerase chain reaction (PCR) reagent and/or one or more of a lysis reagent, primer reagents, a polymerase reagent, deoxynucleotide triphosphate (dNTPs) reagents, a dry reagent, a liquid reagent, and/or a buffer reagent. If the reagent 125 is a dry reagent, the dry reagent may be rehydrated prior to use. However, different and/or additional reagents may be included.

After the sample 122 is added to the sample chamber 102, the sample 103 and the reagent 125 can flow through the respective sample fluidic line 108 and the reagent fluidic line 110 toward the common fluidic line 109 and form a mixture within the common fluidic line 109. Advantageously, the common fluidic line 109 enables the mixture to incubate for a threshold incubation period prior to the mixture flowing to the diagnostic indicator 114. The diagnostic indicator 114 may provide a visual indication as to the presence or absence of the target molecule within the sample 122.

The common fluidic line 109 is at least one of sized or shaped to enable the threshold incubation period, such as including a serpentine channel 126. The incubation period may be approximately five minutes. However, the incubation period may be a different time period depending on the reagent, the target molecule, etc. The common fluidic line 109 is also shown including a passive mixer 127, such as a herringbone mixer 128, that is used to mix the sample 122 and the reagent 125.

In the implementation shown, the sample fluidic line 108 and/or the reagent fluidic line 110 also includes a serpentine channel 126 to allow a threshold incubation period for the sample 122/reagent 125 prior to the sample 122 and/or the reagent 125 mixing within the common fluidic line 109. The serpentine channel 126 of the sample fluidic line 108 may allow an incubation period of between approximately 30 minutes and approximately 45 and the serpentine channel 126 of the common fluidic line 109 may allow an incubation period of approximately 5 minutes. However, different incubation periods may occur as appropriate.

Referring to the pump 116, the pump 116 is used the urge the sample from the sample chamber 102 toward the common fluidic line 109 and to urge the reagent 125 from the reagent reservoir 104 toward the common fluidic line 109. The pump 116 may be a piston pump 129 including a piston 130 that is slidably disposed within a bore 131 that is defined by a body 132 of the testing device 100. A spring 133 may be included that urges the piston 130 in a direction generally indicated by arrow 134 and out of the bore 131.

The testing device 100 also includes a lid 135 that is movable from an open position to a closed position shown in FIG. 1 and is hingabely coupled to the body 132 of the testing device 100 by a hinge 136. The hinge 136 may be a living hinge, a piano hinge, a butt hinge, etc.

In some implementations, as the lid 135 moves from the open position to the closed position, the lid 135 moves the piston 130 within the bore 131, thereby pressurizing the testing device 100 and urging the sample 122 and the reagent 125 through the corresponding fluidic lines 108, 109, 110. To allow the sample chamber 102 and the reagent reservoir 104 to be pressurized by the piston pump 129, in the closed position, the lid 135 seals against corresponding openings 138, 140 of the sample chamber 102 and the reagent reservoir 104, thereby enabling the sample 122 and the reagent 125 to be urged through the fluidic lines 108, 109, 110 and toward the diagnostic indicator 114. While the lid 135 is mentioned sealing against both the sample chamber 102 and the reagent reservoir 104, in other implementations, the reagent reservoir 104 may be a sealed container and, thus, the lid 135 may seal against the opening 138 of the sample chamber 102 to pressurize the testing device 100.

In the implementation shown, a hydrophobic venting membrane 142 covers the opening 140 of the reagent reservoir 104 and may be used to deter the reagent 125 contained within the reagent reservoir 104 from spilling out of the reagent reservoir 104. Additionally, an impermeable membrane 143 may cover the openings 138, 140 of the sample chamber 102 and/or the reagent reservoir 104 prior to use to deter the buffer 124 and/or the reagent 125 from evaporating. The impermeable membrane 143 may be foil and may be pierced prior to a testing operation occurring by, for example, a piercer on the lid 135 or other means.

The heater 118 may be thermoelectric tape and/or a flexible heater and may be positioned to heat at least a portion of the sample fluidic line 108, a portion of the reagent fluidic line 110, and/or a portion of the common fluidic line 109. In some implementations, the heater 118 includes a first heater 144 that is positioned to heat at least a portion of the sample fluidic line 108 and a second heater 146 that is positioned to heat at least a portion of the common fluidic line 109. As such, the heaters 144, 146 may be used to independently control the temperature of the corresponding fluidic lines 108, 109 and its contents. For example, the first heater 144 may heat the sample 122 within the sample fluidic line 108 to approximately 95° C. and the second heater 146 may heat the mixture of the sample 122 and the reagent 125 within the common fluidic line 109 to approximately 65°. Alternatively, one or more of the heaters 144, 146 may be omitted and/or a heater may be provided for the reagent fluidic line 110. A heat sink 148 may also be provided that enables local heating of one of more of the fluidic lines 108, 109, 110 and/or to avoid the sample 122 and/or the reagent 125 from being heated above a threshold temperature.

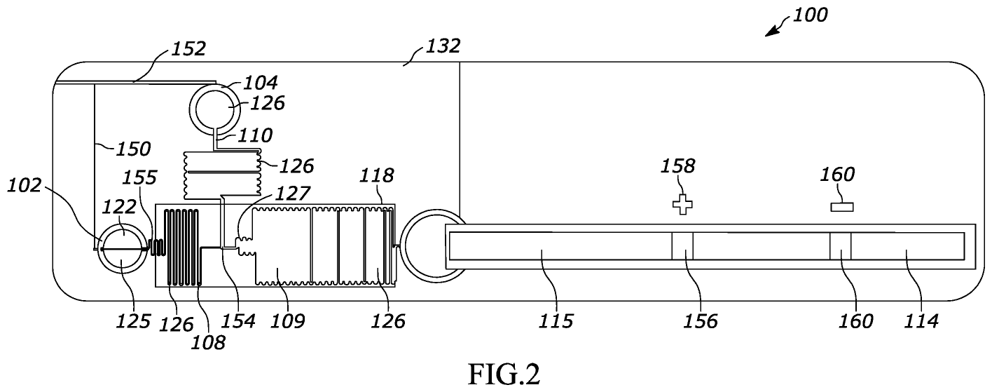

FIG. 2 is a top plan view of an example implementation of a portion of the testing device 100 of FIG. 1. In the implementation shown, the testing device 100 includes the sample chamber 102, the reagent reservoir 104, the diagnostic indicator 114, and the heater 118. Fluidic lines 150, 152 are included that are fluidically coupled to the sample chamber 102 and the reagent reservoir 104 and allow the sample chamber 102 and/or the reagent reservoir 104 to be, for example, pressurized by the pump 116. As shown, the sample fluidic line 108 and the reagent fluidic line 110 are coupled at an intersection 154 where the common fluidic line 109 begins. In the implementation shown, the sample fluidic line 108 also includes a passive mixer 155 that is used to mix the sample 122

Referring to the serpentine channel 126 of the different fluidic lines 108, 109, 110, in the implementation shown, the serpentine channel 126 of the sample fluidic line 108 is partially positioned over top of the heater 118, the serpentine channel 126 of the reagent fluidic line 110 is spaced from the heater 118, and the serpentine channel 126 of the common fluidic line 109 is positioned over top of the heater 118. The serpentine channels 126 may be designed so the sample 122 is substantially simultaneously mixed and heated to a threshold temperature for a threshold period of time. Depending on the reagent 125 being used and/or the target molecule being detected, more or less of the respective fluidic lines 108, 109, 110 may be positioned over top of the heater 118.

The testing device 100 of FIG. 2 also shows the coupling between the common fluidic line 109 and the diagnostic indicator 114 including the litmus-style display 115. The litmus-style display 115 is shown including a first line 156 adjacent a positive sign 158 and a second line 160 adjacent a negative sign 162. The first line 156 and the second line 160 being present after a testing operation may indicate that the target molecule is present in the sample 122 and only the second line 160 being present after a testing operation may indicate that the target molecule is not present in the sample 122.

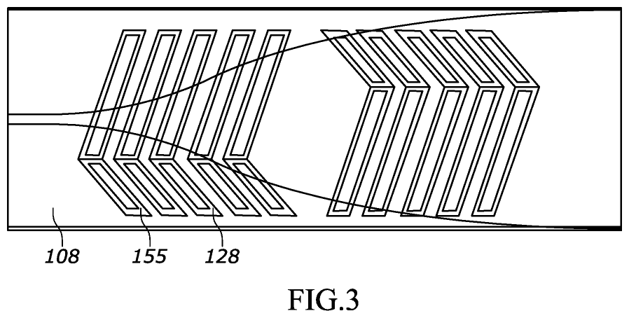

FIG. 3 is a detailed view of the passive mixer 155 of the sample fluidic line 108 of FIG. 2. In the implementation shown, the passive mixer 155 includes the herringbone mixer 128 and is used to mix the sample 122 and the buffer 124.

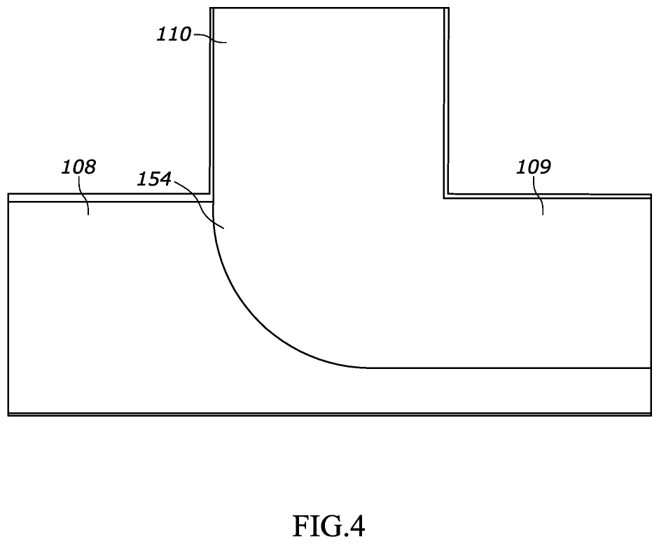

FIG. 4 is a detailed view of the intersection 154 between the sample fluidic line 108 and the reagent fluidic line 110 of the testing device 100 of FIG. 2. In the implementation shown, the fluidic lines 108, 110 intersect at approximately 90° and the sample fluidic line 108 and the common fluidic line 109 are substantially coaxial. However, the fluidic lines 108, 109, 110 may be positioned in any position relative to one another.

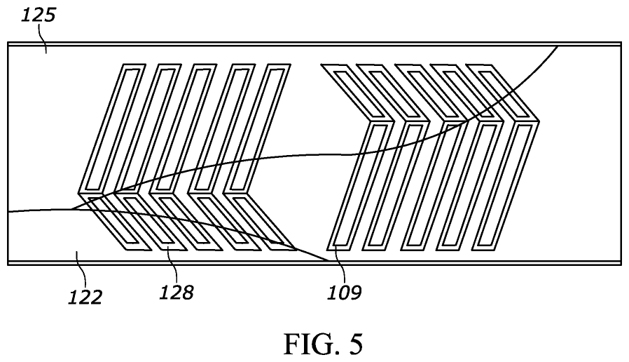

FIG. 5 is a detailed view of the passive mixer 127 of the common fluidic line 109 of the testing device 100 of FIG. 2. In the implementation shown, the passive mixer 127 includes the herringbone mixer 128 and is used to mix the sample 122 and the reagent 125.

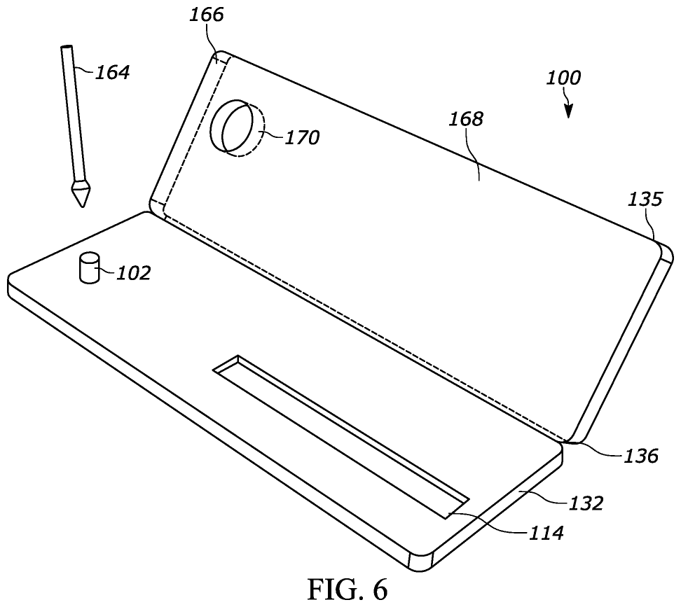

FIG. 6 is an isometric view of another implementation of the testing device 100 of FIG. 1 and a swab 164 that is used to obtain a sample from an individual. In the implementation shown, the testing device 100 includes the sample chamber 102, the diagnostic indicator 114, and the lid 135 that is hingably coupled to the body 132 by the hinge 136. The lid 135 includes a peripheral lip 166 that defines a cavity 168 that may receive at least a portion of the body 132 of the testing device 100 when the lid 135 is in the closed position. The lid 135 also includes a collar 170 that is positioned to at least partially surround and sealingly engage the sample chamber 102 when the lid 135 is in the closed position to allow the testing device 100 to be pressurized by the pump 116.

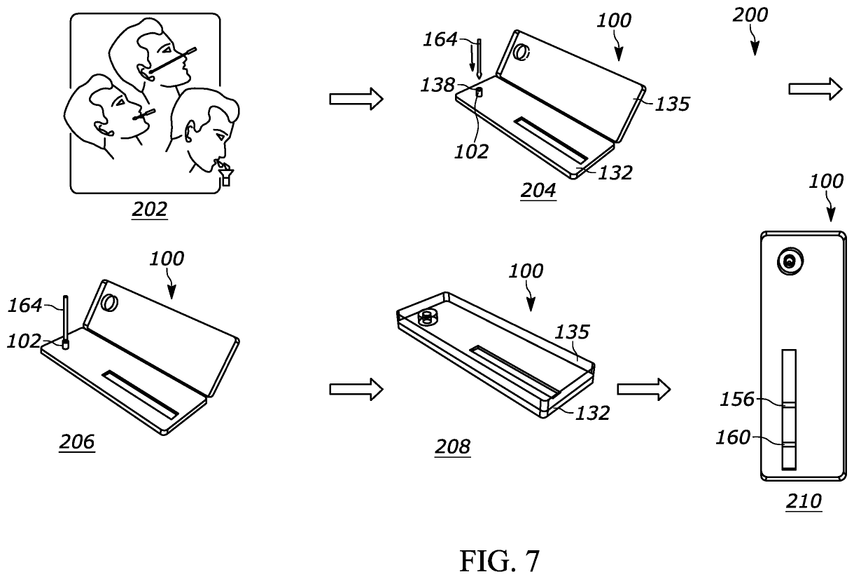

FIG. 7 is a process diagram 200 including processes used to perform a testing operation using the testing device 100 in accordance with the teachings of this disclosure. At reference number 202, the sample 122 is obtained. The sample may be obtained using a nasal swab, a nasopharyngeal swab, a throat swab, and/or obtaining a saliva sample. At reference number 204, the swab 164 carrying the sample 122 is aligned with the opening 138 of the sample chamber 102 and, at reference number 206, a distal end of the swab 164 having the sample 122 is positioned within the sample chamber 102. The swab 164 may remain in the sample chamber 102 for a threshold amount of time. For example, the swab 164 with the sample 122 may remain in the sample chamber 102 for approximately 2 minutes. However, the swab 164 may remain in the sample chamber 102 for different amounts of time.

At reference number 208, the lid 135 is shown in the closed position. Closing the lid 135 seals the sample chamber 102 and may cause the pump 116 to pressurize the testing device 100 and urge the sample 122 and the reagent 125 toward the common fluidic line 109. Closing the lid 135 may also initiate the heater 118 heating one or more of the fluidic lines 108, 109, 110 to the threshold temperature. In some implementations, the assay will start automatically when the lid 135 is closed. At reference number 210, the lines 156, 160 are shown being displayed on the diagnostic indicator 114, indicating the presence of the target molecule. In some implementations, the results of the testing operation are complete after a threshold amount of time such as, for example, 45 minutes.

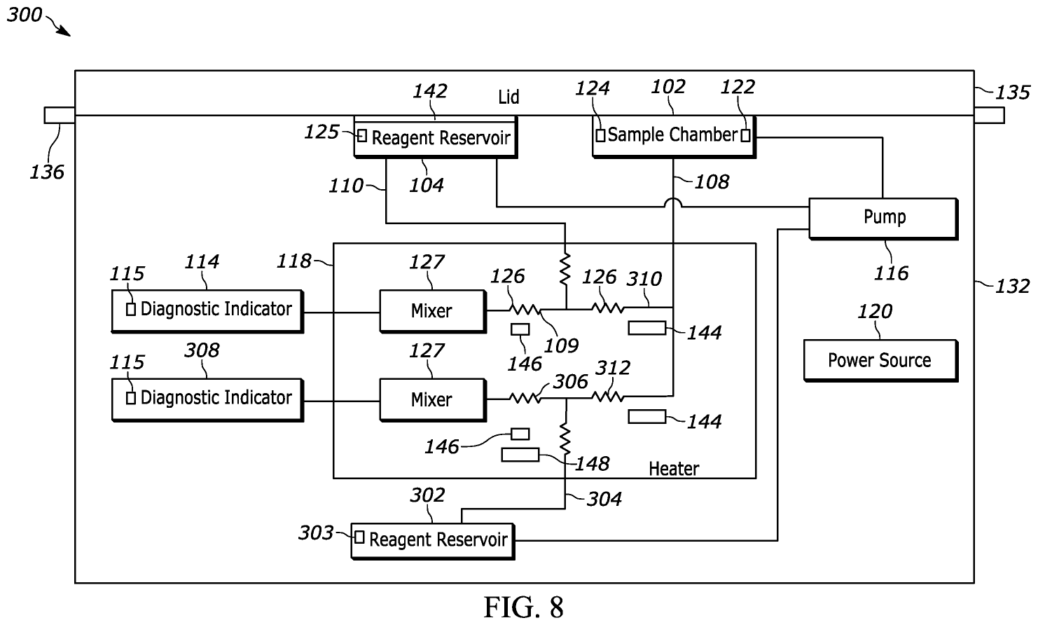

FIG. 8 is a schematic diagram of an implementation of another testing device 300 in accordance with the teachings of this disclosure. The testing device 300 of FIG. 8 is similar to the testing device of FIG. 1. However, in contrast, the testing device 300 of FIG. 8 is able to detect for the presence of multiple target molecules.

To do so, the testing device 300 includes a second reagent reservoir 302 that contains a second reagent 303 used to determine the presence of a second target molecule in the sample 122. The testing device 300 also includes a second reagent fluidic line 304, a second common fluidic line 306, and a second diagnostic indicator 308. The second reagent fluidic line 304 fluidically couples the second reagent reservoir 302 and the second common fluidic line 306 and the second common fluidic line 306 is coupled to the second diagnostic indicator 308. The sample fluidic line 108 also includes a first-sub portion 310 that fluidically couples the sample chamber 102 and the first common fluidic line 109 and a second-sub portion 312 that fluidically couples the sample chamber 102 and the second common fluidic line 306. In some implementations, one or more of the heaters 144, 146 of the testing device 300 can operate independently of one another and/or on different time regimes and/or schedules. As such, the testing device 300 may be used to perform different side-by-side experiments. For example, for a first experiment (e.g., a polymerase chain experiment) performed by the testing device 300, the fluidic lines 126 and/or 310 and/or the associated sample 122 and/or reagent 125 may be heated to a first threshold temperature heated for a first threshold period of time and, for a second experiment (e.g., a Loop-mediated Isothermal Amplification experiment) performed by the testing device 300, the fluidic lines 306 and/or 312 and/or the associated sample 122 and/or reagent 303 may be heated to a second threshold temperature for a second threshold period of time. While the testing device 300 is able to test for the presence of two target molecules, the testing device 300 may be able to test for any number of target molecules. For example, when configured to test for five target molecules, the testing device 300 may include five reagent reservoirs, five diagnostic indicators, and associated fluidic lines.

To perform a testing operation using the testing device 300 of FIG. 8, the sample 122 is added to the sample chamber 102. The sample 103 and the reagents 125, 303 can flow through the respective sample fluidic lines 108, 310, 312 and the reagent fluidic lines 110, 304 toward the corresponding common fluidic lines 109, 306 and form a mixture within the common fluidic lines 109, 306. The common fluidic lines 109, 306 may be adapted to enable a similar, the same, or different incubation periods prior to the mixtures flowing to the corresponding diagnostic indicators 114, 308, where the results of the testing operation may be displayed.

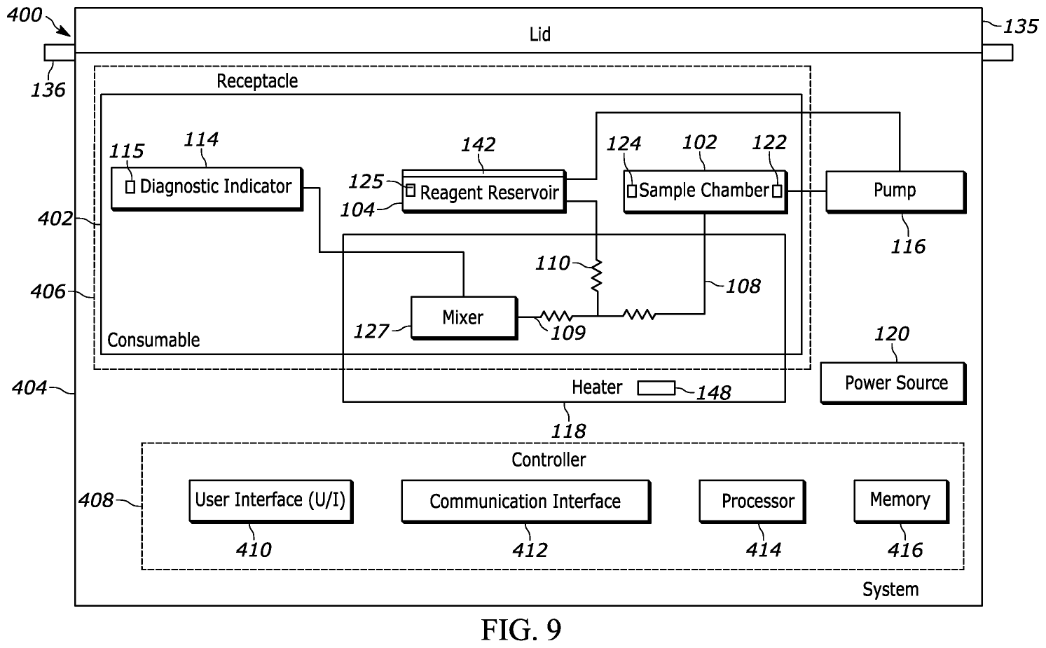

FIG. 9 is a schematic diagram of an implementation of another testing device 400 in accordance with the teachings of this disclosure. The testing device 400 of FIG. 9 is similar to the testing device 100 of FIG. 1. However, in contrast, the testing device 400 of FIG. 9 includes a consumable 402 and a system 404 including a receptacle 406 that receives the consumable 402. The consumable 402 is adapted for one-time use while the system 404 and its components are intended for repeated and/or consistent use. Thus, the testing device 400 of FIG. 9 may be usable in a point-of-care setting or in any area where frequent testing is performed.

In the implementation shown, the consumable 402 includes the sample chamber 102, the reagent reservoir 104, the sample fluidic line 108, the reagent fluidic line 110, and the common fluidic line 109. The system 404 includes the pump 116, the heater 118, a controller 408, and the power source 120. The pump 116 may be a piston pump, a syringe pump, a peristaltic pump, an electronic pump, a piezoelectric pump, and/or a diaphragm pump. However, other types of fluid transfer devices may be used. The controller 408 is electrically and/or communicatively coupled to the pump 116, the heater 118, and the power source 120 to perform various functions as disclosed herein. When the consumable 402 is received within the receptacle 406 of the system 404, the pump 116 is fluidically coupled to the sample chamber 102 and the reagent reservoir 104 and the heater 118 is positioned to heat one or more of the sample fluidic line 108, the reagent fluidic line 110, or the common fluidic line 109.

Referring to the controller 408, in the implementation shown, the controller 408 includes a user interface 410, a communication interface 412, one or more processors 414, and a memory 416 storing instructions executable by the one or more processors 414 to perform various functions including the disclosed implementations. The user interface 410, the communication interface 412, and the memory 416 are electrically and/or communicatively coupled to the one or more processors 414.

In an implementation, the user interface 410 receives input from a user and provides information to the user associated with the operation of the system 404 and/or an analysis taking place. For example, the user interface 410 can provide a visual display to indicate the presence or an absence of the target molecule within the sample. However, the user interface 410 can display additional or different prompts and/or information. The user interface 410 may include a touch screen, a display, a key board, a speaker(s), a mouse, a track ball, and/or a voice recognition system. The touch screen and/or the display may display a graphical user interface (GUI).

In an implementation, the communication interface 412 is adapted to enable communication between the system 404 and a remote system(s) (e.g., computers) using a network(s). The network(s) may include the Internet, an intranet, a local-area network (LAN), a wide-area network (WAN), a coaxial-cable network, a wireless network, a wired network, a satellite network, a digital subscriber line (DSL) network, a cellular network, a Bluetooth connection, a near field communication (NFC) connection, etc. Some of the communications provided to the remote system may be associated with analysis results, etc. generated or otherwise obtained by the system 404. Some of the communications provided to the system 404 may be associated with the presence or absence of a target molecule, a diagnostics procedure, an analysis operation, patient records, and/or a protocol(s) to be executed by the system 404.

The one or more processors 414 and/or the system 404 may include one or more of a processor-based system(s) or a microprocessor-based system(s). In some implementations, the one or more processors 414 and/or the system 404 includes one or more of a programmable processor, a programmable controller, a microprocessor, a microcontroller, a graphics processing unit (GPU), a digital signal processor (DSP), a reduced-instruction set computer (RISC), an application specific integrated circuit (ASIC), a field programmable gate array (FPGA), a field programmable logic device (FPLD), a logic circuit and/or another logic-based device executing various functions including the ones described herein.

The memory 416 can include one or more of a semiconductor memory, a magnetically readable memory, an optical memory, a hard disk drive (HDD), an optical storage drive, a solid-state storage device, a solid-state drive (SSD), a flash memory, a read-only memory (ROM), erasable programmable read-only memory (EPROM), electrically erasable programmable read-only memory (EEPROM), a random-access memory (RAM), a non-volatile RAM (NVRAM) memory, a compact disc (CD), a compact disc read-only memory (CD-ROM), a digital versatile disk (DVD), a Blu-ray disk, a redundant array of independent disks (RAID) system, a cache and/or any other storage device or storage disk in which information is stored for any duration (e.g., permanently, temporarily, for extended periods of time, for buffering, for caching).

FIG. 10 is a schematic diagram of an implementation of another testing device 500 in accordance with the teachings of this disclosure. The testing device 500 of FIG. 10 is similar to the testing device 400 of FIG. 9. However, in contrast, the system 404 of FIG. 10 includes an imaging system 502 and the consumable 402 of FIG. 10 includes a fluidic channel or flow cell 504. The fluidic channel and/or flow cell 504 may be referred to as a viewing chamber. The imaging system 502 may be a fluorescence spectrophotometer including an objective lens and/or a solid-state imaging device. The imaging system 502 may additionally or alternatively be a fluorescent microscope or may be part of a phone or smart device and/or may include a magnifying component, a fluorescent component, and/or a detector component.

The testing device 500 also includes a droplet generator 180 that includes a fluidic line 182 coupled to the common fluid line 109. The fluidic line 182 may additionally or alternatively be coupled to either or both of the fluidic lines 108, 112. The fluidic line 182 may contain a continuous non-aqueous phase liquid 184. The continuous non-aqueous phase liquid 184 may be a continuous non-aqueous phase oil with surfactant. The fluidic line 182 meets the common line 109 and allows the liquid 184 to form a droplet including the sample 122 and the reagent 125. The liquid 184 may mix, surround, and/or pinch off a droplet of the sample 122 and the reagent 125 that may be stabilized by surfactant of the liquid 184. The liquid 184 and the reagent 125 may form a fluorescent droplet emulsion in some implementations.

In operation, a mixture of the sample 122 and the reagent 125 is urged into the flow cell 504 and the imaging system 502 excites one or more identifiable labels (e.g., a fluorescent label) that are attached to the reagent 125 and thereafter obtains image data for the identifiable labels. The labels may be excited by incident light and/or a laser and the image data may include one or more colors emitted by the respective labels in response to the excitation. The image data (e.g., detection data) may be analyzed by the system 404 to determine the presence of a target molecule. The imaging system 502 may also be able to individually detect (via magnification component) droplets, excite any fluorescent reagents (via fluorescent component), and image the droplets for counting purposes as an example.

Further, while several examples have been disclosed herein, any features from any examples may be combined with or replaced by other features from other examples. Moreover, while several examples have been disclosed herein, changes may be made to the disclosed examples without departing from the scope of the claims.

1. A testing device, comprising:
a sample chamber to receive a sample;
a reagent reservoir containing a reagent used to determine a presence of a target molecule in the sample;
a sample fluidic line, a reagent fluidic line, and a common fluidic line, the sample fluidic line being fluidically coupled to the sample chamber and the common fluidic line and the reagent fluidic line being fluidically coupled to the reagent reservoir and to the common fluidic line; and
a diagnostic indicator coupled to the common fluidic line,
wherein the sample and the reagent flow through the respective sample fluidic line and the reagent fluidic line toward the common fluidic line and form a mixture within the common fluidic line and wherein the common fluidic line enables a threshold incubation period of the mixture prior to the mixture flowing to the diagnostic indicator.

2-3. (canceled)

4. The testing device of claim 1, wherein the reagent comprises one or more of a Loop-mediated Isothermal Amplification (LAMP) reagent, a polymerase chain reaction (PCR) reagent, a lysis reagent, a primer reagent, a polymerase reagent, a deoxynucleotide triphosphate (dNTPs) reagent, dry reagent, liquid reagent, and a buffer reagent.

5-11. (canceled)

12. The testing device of claim 1, wherein the common fluidic line is at least one of sized or shaped to enable the threshold incubation period.

13. The testing device of claim 1, wherein the common fluidic line comprises a serpentine channel that enables the threshold incubation period.

14. (canceled)

15. The testing device of claim 1, wherein the common fluidic line comprises a passive mixer.

16. The testing device of claim 15, wherein the passive mixer comprises a herringbone mixer.

17. The testing device of claim 1, further comprising a heater positioned to heat one or more of the sample fluidic line, the reagent fluidic line, or the common fluidic line.

18. The testing device of claim 17, wherein the heater comprises at least one of thermoelectric tape or a flexible heater.

19. (canceled)

20. The testing device of claim 17, wherein the heater is positioned to heat at least a portion of the sample fluidic line, a portion of the reagent fluidic line, and a portion of the common fluidic line.

21. (canceled)

22. The testing device of claim 1, further comprising a first heater positioned to heat at least a portion of the sample fluidic line and a second heater positioned to heat a portion of the common fluidic line.

23. The testing device of claim 1, further comprising a pump to urge the sample from the sample chamber toward the common fluidic line and to urge the reagent from the reagent reservoir toward the common fluidic line.

24. The testing device of claim 23, wherein the pump comprises a piston pump.

25. The testing device of claim 24, further comprising a body defining a bore, the piston pump comprising a piston and the bore, the piston being slidably disposed with the bore.

26. The testing device of claim 25, further comprising a lid hingably coupled to the body and movable between an open position and a closed position, the lid to move the piston within the bore when the lid moves toward the closed position.

27. The testing device of claim 26, wherein the sample chamber and the reagent reservoir comprise corresponding openings and wherein the lid seals against the openings when the lid is in the closed position to allow the sample chamber and the reagent reservoir to be pressurized by the piston pump.

28. The testing device of claim 26, wherein the sample chamber comprises a corresponding opening and wherein the lid seals against the opening when the lid is in the closed position to allow the sample chamber to be pressurized by the piston pump.

29. The testing device of claim 1, further comprising a hydrophobic venting membrane covering an opening of the reagent reservoir.

30-32. (canceled)

33. The testing device of claim 1, further comprising:
a second reagent reservoir containing a second reagent used to determine a presence of a second target molecule in the sample;
a second reagent fluidic line and a second common fluidic line, the sample fluidic line being fluidically coupled to the sample chamber and the second common fluidic line and the second reagent fluidic line being fluidically coupled to the second reagent reservoir and to the second common fluidic line; and
a second diagnostic indicator coupled to the second common fluidic line.

34. The testing device of claim 1, further comprising a consumable comprising the sample chamber, the reagent reservoir, the sample fluidic line, the reagent fluidic line, and the common fluidic line, further comprising a system comprising:
a receptacle to receive the consumable;
a pump fluidically couplable to one or more of the sample chamber, the reagent reservoir, the sample fluidic line, or the reagent fluidic line; and
a heater positioned to heat one or more of the sample fluidic line, the reagent fluidic line, or the common fluidic line,
wherein the pump urges the sample from the sample chamber toward the common fluidic line and urges the reagent from the reagent reservoir toward the common fluidic line.

35-43. (canceled)

44. The testing device of claim 1, further comprising a droplet generator comprising a fluidic line coupled to one or more of the sample fluidic line, the reagent fluidic line, and the common fluidic line.

45-48. (canceled)
